(19) United States
(12) Patent Application Publication
(10) Pub. No.: US 2026/0272788 A1
(43) Pub. Date: Sep. 17, 2026

(54) MIXTURE, POLYMER, DENTAL POLYMER FOR 3D PRINTING AND MEDICAL PRODUCT THEREOF

(71) Applicant: PRO3DURE MEDICAL GMBH, Iserlohn (DE)

(72) Inventors: Martin KLARE, Dortmund (DE); Frank GISCHER, Iserlohn (DE)

(73) Assignee: PRO3DURE MEDICAL GMBH, Iserlohn (DE)

(21) Appl. No.: 19/165,123

(22) PCT Filed: Mar. 13, 2024
(86) PCT No.: PCT/EP2024/056731

§ 371 Date: Sep. 14, 2025

(30) Foreign Application Priority Data
Mar. 13, 2023 (EP) 23161615.2

(51) Int. Cl. A61K 6/60; A61K 6/889; C08K 5/3415

(52) U.S. Cl. CPC A61K 6/60; A61K 6/889; C08K 5/3415

(57) ABSTRACT

The present invention relates to a mixture, for 3D printing, particularly a resin for 3D printing comprising—a monomer, oligomer or polymer, or a mixture thereof, comprising at least one acrylate or methacrylate subunit, and—a lactam, characterized in that the lactam concentration is 0.001 to 0.3 wt %, particularly the lactam concentration is 0.001 to 0.15 wt %, more particularly the lactam concentration is 0.01 to 0.1 wt %. Furthermore, the present invention relates to a polymer, in particular a dental polymer or an otoplastic polymer comprising polymerised monomers and/or oligomers or polymers and a lactam, as well as a medical product comprising said polymer.

### FIELD

The invention relates to a mixture, polymer, dental polymer for 3D printing with reduced biofilm adhesion as well as a medical product thereof.

### BACKGROUND

Surfaces of 3D printed objects are microstructured due to the manufacturing process. Thus, in the case of image projection systems, individual pixels are exposed and imaged on a resin surface. As a result, lateral structuring (XY direction) is created. Often, generative manufacturing processes continue to be layering processes. Accordingly, geometries in the Z direction, for example, can only be imaged in steps, with the effect that surface structuring also occurs in the Z direction. It is known to the skilled person that surface structuring in particular has a significant influence on biofilm adhesion and can lead to plaque build-up and caries in the dental field. Furthermore, 3D printed microfluidic devices are increasingly used in medical diagnostics. These often have fine channel structures that can become clogged, for example by biofilms, and thus influence diagnostics. Especially in the region of microfluidics, a defined shape of the internal channel structures is of paramount importance to keep the desired flow properties in the member controllable and thus adjustable. Due to the technology involved, however, a channel with a circular cross-section built perpendicular to the direction of image projection can only be constructed in layers (stair steps). In order to bond the individual layers together during construction (intra layer curing), a so-called overcuring process must take place. This means that in the case of a 100 micrometer thick layer, the penetration depth of the polymerization must take place at >100 micrometers to create a bonding between the layers. The skilled person knows that when developing/adjusting a 3D printing resin, about 1.5 times overcuring should be achieved. This adjustment of the polymerization characteristics of a 3D printing resin can be described by means of the two variables critical energy (Ec value) and depth of cure (Dp value). The Ec value characterizes the threshold value of radiation energy at which polymerization begins, and the Dp value characterizes the penetration depth of the radiation. In most cases, these two variables can only be adjusted in dependence on each other. According to the state of the art, this adjustment of the resin characteristics is nowadays performed, by the selected photoinitiator system (or systems), by so-called UV blockers, optical brighteners and, in some cases, by polymerization stabilizers. Various problems arise from the complex interaction of all these components with each other. On the one hand, components such as the photoinitiator TPO comprise carcinogenic and fertility-damaging effects above certain concentration ranges and are therefore listed in the CFR today. On the other hand, UV blockers and optical brighteners such as 2-(2H-benzotriazol-2-yl) p-cresol change the optical behavior of the generated members under radiation (e.g. afterglow in UV light) and also the polymerization kinetics in the cured layers. Accordingly, altered chemical-physical properties can occur as a result of e.g. shorter polymer chains. In addition, intra-layer curing is also changed by this, and in individual cases this can lead to a lack of bonding between the layers or to incomplete post-curing of the members in the volume. Furthermore, the above-mentioned components always have a common influence on Dp and Ec values.

For these reasons, it is desirable to provide a material with reduced biofilm adhesion and at the same time be able to adjust the mechanical properties in particular the Dp and Ec values, for 3D printing more easily (independently of each other).

It is thus the objective of the present invention to provide such a mixture for 3D printing, particularly a resin for 3D printing with reduced biofilm adhesion and improved Ec and Dp values. This objective is solved by the subject matter of the independent claim 1, wherein certain embodiments are stated in subclaims 2-12. Furthermore, it is the objective to provide a polymer produced by 3D printing according to independent claim 13, wherein certain embodiments of the polymer produced by 3D printing are stated in subclaim 14. Additionally, the invention provides a dental polymer or otoplastic polymer according to independent claim 15. Finally, the invention provides a medical product according to independent claim 16.

### SUMMARY OF THE INVENTION

A first aspect of the invention relates to a mixture for 3D printing, particularly a resin for 3D printing comprising
a monomer, oligomer or polymer, or a mixture thereof, comprising at least one acrylate or methacrylate subunit, and
a lactam,
   characterized in that the lactam concentration is 0.001 to 0.3 wt %, particularly the lactam concentration is 0.005 to 0.15 wt %, more particularly the lactam concentration is 0.01 to 0.1 wt %.

A second aspect of the invention relates to a polymer, particularly produced by 3D printing using the resin for 3D printing according to claims 1 to 10, wherein the polymer comprises
polymerised monomers, oligomers or polymers, or a mixture thereof, comprising at least one acrylate or methacrylate subunit, and
a lactam,
   characterized in that the lactam concentration is 0.001 to 0.3 wt %, particularly the lactam concentration is 0.005 to 0.15 wt %, more particularly the lactam concentration is 0.01 to 0.1 wt %, and wherein the monomer, oligomer, polymer and lactam are defined as above. The lactam concentration can for example comprise any lactam concentration according to the first aspect of the invention.

A third aspect of the invention relates to a dental polymer or an otoplastic polymer, wherein the dental polymer or otoplastic polymer is a polymer according to the first and/or second aspect of the invention.

A fourth aspect of the invention relates to a medical product according to the first, second and third aspect of the invention.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

“And/or” where used herein is to be taken as specific recitation of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The formulae of the present specification follow the convention of organic chemistry to not show hydrogen atoms on carbon scaffolds. Carbon is tetravalent and bonds not shown are assumed to be hydrogen unless shown otherwise.

The term mixture in the context of the present specification relates to a composition of at least one of a monomer to be polymerised, an oligomer and a polymer, and a lactam. The monomer to be polymerised, oligomer, polymer and lactam may be present as solid, powder or liquid.

The term alkyl in the context of the present specification relates to a substituted or unsubstituted, saturated linear, branched or (partially or completely) cyclic hydrocarbon. The A substituted alkyl may be substituted with further alkyl residues, hydroxyl moieties, amine moieties and/or aromatic moieties or with other moieties as stated herein.

The term C₁-C₂₂ alkyl in the context of the present specification relates to a substituted or unsubstituted, saturated linear or branched hydrocarbon having 1 to 22 carbon atoms. The C₁-C₂₂ alkyl or any alkyl chains included in this range may be further substituted with further alkyl residues, hydroxyl moieties, amine moieties and/or aromatic moieties or with other moieties as stated herein.

The term aryl in the context of the present specification relates to a substituted or unsubstituted cyclic aromatic C₅-C₁₀ hydrocarbon. In particular, the term aryl in the context of the present specification relates to a substituted or unsubstituted cyclic aromatic C₅-C₆ hydrocarbon. Examples of C₅-C₁₀ aryl include, without being restricted to, phenyl and naphthyl. Substituted aryl moieties may be substituted with one or more moieties selected from C₁₋₄ alkyl, —OH, —NH₂.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂ (iso-propyl, i-pr), but is butyl —C₄H₉, —(CH₂)₃CH₃, —CHCH₃CH₂CH₃, —CH₂CH(CH₃)₂ or —C(CH₃)₃.

The term oligomer in the context of the present specification relates to a macro molecule comprising up to 30 structurally identical or similar units, particularly a macro molecule comprising 10 to 30 structurally identical or similar units.

The term polymer in the context of the present specification relates to a macro molecule comprising at least 31 structurally identical or similar units. The term polymer is further defined by a viscosity of at least 250 Pas at 60° C.

The term EO in the context of the present specification relates to ethylene oxide. The number in brackets, e.g. EO (2-60), relates to the number of ethylene oxide units.

The term PO in the context of the present specification relates to propylene oxide. The number in brackets, e.g. PO (2-60), relates to the number of propylene oxide units.

The term wt % in the context of the present specification relates to the weight percentage relative to the total mass (100%) of the mixture.

### DETAILED DESCRIPTION

A first aspect of the invention relates to a mixture for 3D printing, particularly a resin for 3D printing comprising
a monomer, oligomer or polymer, or a mixture thereof, comprising at least one acrylate or methacrylate subunit, and
a lactam,
   characterized in that the lactam concentration is 0.001 to 0.3 wt %, particularly the lactam concentration is 0.005 to 0.15 wt %, more particularly the lactam concentration is 0.01 to 0.1 wt %.

Acrylates and methacrylates are polymerised through radical polymerisation. It is, however, known by a person skilled in the art that also cationic polymerisation can be used in case of using epoxide monomers or epoxy resins as e.g. stated in WO 2020/229444, as well as a mixture of both. In case of radical polymerisation, at least 20% of a monomer with an acrylate or methacrylate subunit are required.

In certain embodiments, the lactam concentration is 0.001 to 0.3 wt %.

At lactam concentrations of up to 0.3 wt % the polymer derived from the mixture shows a decreased biofilm adhesion

In certain embodiments, the lactam concentration is 0.001 to 0.25 wt %. In certain embodiments, the lactam concentration is 0.001 to 0.2 wt %. In certain embodiments, the lactam concentration is 0.001 to 0.15 wt %. In certain embodiments, the lactam concentration is 0.005 to 0.15 wt %.

In certain embodiments, the lactam concentration is 0.001 to 0.1 wt %. In certain embodiments, the lactam concentration is 0.01 to 0.1 wt %.

In certain embodiments according to the first aspect of the invention, the lactam concentration is 0.03 to 0.06 wt %.

By using lactam, the Ec value is increased and the Dp value is decreased, in a linear manner, providing an ideal way to regulate the curing characteristics of the 3D printing mixture, allowing for high resolution printing materials and long-term stability of the printing material, while the biofilm adhesion remains low.

In certain embodiments, the lactam is a γ-lactam or furanone, particularly a γ-lactam.

In certain embodiments, the lactam is a compound of formula (1) or (2)

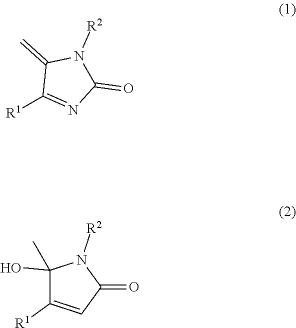

wherein R¹ is a substituted or unsubstituted aryl, wherein the substituted aryl is substituted with Z¹, wherein Z¹ is selected from the group consisting of C₁-C₃ alkyl F, Cl, and Br; and R² is selected from H and the group consisting of —C₁₋₃-alkyl-N⁺Me₃, particularly R² is H.

In certain embodiments, the monomer is of formula (3a), the oligomer is of formula (3b) and the polymer is of formula (3c),

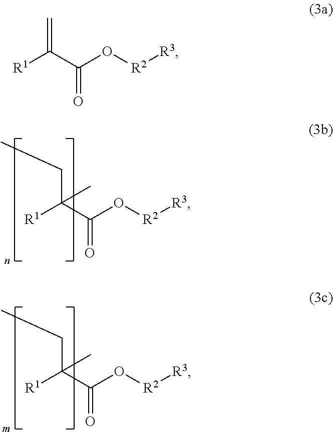

wherein n is 2 to 30 and m is 31 to 100,
R¹ is —H or —CH₃,
R² is selected from the group consisting of
   —C₁— bis C₂₂-alkyl-, -allyl-, -vinyl-,
   —C₁-bis C₂₂-alkyl-O-C1- bis C₂₂-alkyl-, wherein the —C₁₋₂₂-alkyl- is further substituted, particularly wherein the —C₁₋₂₂-alkyl- is further substituted with 1 to 4 acrylate or 1 to 4 methacrylate moieties,
   —[(CH₂)ₑ—O-]_{f}-C₁₋₆-alkyl-[(CH₂)ₑ—O-]_{f}, wherein the —C₁₋₆-alkyl- is further substituted, particularly wherein the —C₁₋₆-alkyl- is further substituted with 1 to 4-[(CH₂)ₑ—O-]_{f}-acrylate moieties or 1 to 4-[(CH₂)ₑ—O-]_{f} methacrylate moieties, and wherein e is 1 to 3 and f is 2 to 9,

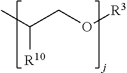

wherein j is 1 to 200, and R¹⁰ is selected from the group consisting of —H, —C₁₋₃-alkyl, —O—C₁₋₃-alkyl, —OH or -phenol,
—(C₁₋₃-alkyl)-O(C═O)NH—R¹¹—NH—C(═O)O—, wherein R¹¹ is a linker system consisting of the group aliphatic or aromatic moieties or a mixture thereof,

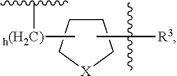

wherein X is O or CH and h is 0 or 1,

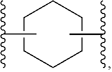

—Ph-Y—(C₁₋₃-alkyl)ₖ- wherein Y is S, O or CH₂ and k is 0 or 1 and wherein the C₁₋₃-alkyl may be further substituted with a moiety selected from the group consisting of —OH and NH₂,

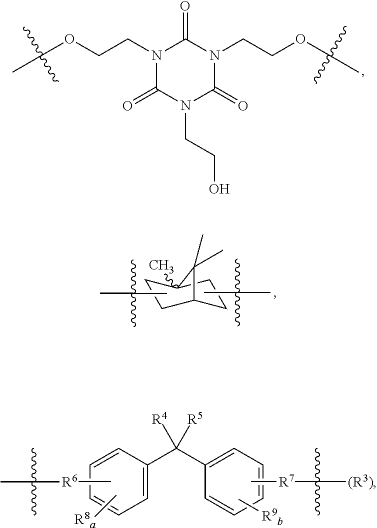

wherein
R⁴ and R⁵ are independently from each other selected from the group consisting of H, —C₁₋₄-alkyl, —CF₃ and phenyl, or
R⁴ and R⁵ form a ring structure consisting of 4 to 8 carbon atoms, particularly R⁴ and R⁵ are independently from each other selected from the group consisting of H and —C₁₋₄-alkyl
R⁶ and R⁷ are selected from —[(CH₂)ₚ—O-]_{q}-, and —O—[(CH₂)ᵥ—O-]_{w}-, wherein p and v are between 1 and 4, particularly m, p and v are between 1 and 3, q is between 1 and 4 and w is between 1 and 200, particularly w is between 1 and 60, more particularly w is between 1 and 30,
Rₐ⁸ and R_{b}⁹ are independently from each other selected from the group consisting of —C₁₋₄-alkyl and phenyl,
a is 0 or 1, particularly a is 0
b is 0 or 1, particularly b is 0 and
R³ is selected from the group consisting of —H, acrylate and methacrylate.

In certain embodiments, R² is selected from
—C₁— bis C₂₂-alkyl-, -allyl-, -vinyl-,
—(C₁₋₃-alkyl)-O(C═O)NH—R¹¹—NH—C(═O)O—, wherein R¹¹ is a linker system consisting of the group aliphatic or aromatic moieties or a mixture thereof,
—Ph—Y—(C₁₋₃-alkyl)ₖ- wherein Y is S, O or CH₂ and k is 0 or 1 and wherein the C₁₋₃-alkyl may be further substituted with a moiety selected from the group consisting of —OH and NH₂,

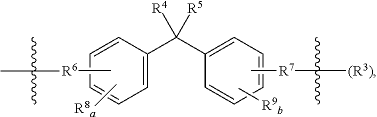

wherein
R⁴ and R⁵ are independently from each other selected from the group consisting of
H, —C₁₋₄-alkyl, —CF₃ and phenyl, or
R⁴ and R⁵ form a ring structure consisting of 4 to 8 carbon atoms, particularly R⁴ and R⁵ are independently from each other selected from the group consisting of H and —C₁₋₄-alkyl
R⁶ and R⁷ are selected from —[(CH₂)ₚ—O-]_{q}-, and —O—[(CH₂)ᵥ—O-]_{w}-, wherein p and v are between 1 and 4, particularly m, p and v are between 1 and 3, q is between 1 and 4 and w is between 1 and 200, particularly w is between 1 and 60, more particularly w is between 1 and 30
Rₐ⁸ and R_{b}⁹ are independently from each other selected from the group consisting of —C₁₋₄-alkyl and phenyl,
a is 0 or 1, particularly a is 0
b is 0 or 1, particularly b is 0.

In certain embodiments, R² is selected from
—C₁-bis C₂₂-alkyl-, particularly C₁₋₁₃-alkyl,
—(C₁₋₃-alkyl)-O(C═O)NH—R¹¹—NH—C(═O)O—, wherein R¹¹ wherein R¹¹ is a linker system consisting of the group aliphatic or aromatic moieties or a mixture thereof, and

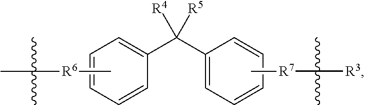

wherein
R⁴ and R⁵ are independently from each other selected from the group consisting of H, —C₁₋₄-alkyl,
R⁶ and R⁷ are selected from —[(CH₂)ₚ—O-]_{q}-, and —O—[(CH₂)ᵥ—O-]_{w}-, wherein p and v are between 1 and 4, particularly m, p and v are between 1 and 3, q is between 1 and 4 and w is between 1 and 200, particularly w is between 1 and 60, more particularly w is between 1 and 30.

In certain embodiments, R⁶ and R⁷ are selected from —O—[(CH₂)ᵥ—O-]_{w}-, wherein v is between 1 and 4, particularly v is between 1 and 3, and w is between 1 and 200, particularly w is between 1 and 60, more particularly w is between 1 and 30.

In certain embodiments, R¹¹ is selected from the group consisting of C₁₋₁₅ alkyl, and substituted or unsubstituted C₆ aryl, particularly R¹¹ is selected from the group consisting of C₁₋₁₃ alkyl, and substituted or unsubstituted C₆ aryl.

In certain embodiments, the monomer, oligomer or polymer is selected from the group consisting of methyl diacrylate, ethyl diacrylate, propyl diacrylate, butyl diacrylate, lauryl diacrylate, palmitoyl diacrylate, stearyl diacrylate, behenyl diacrylate, pentyl diacrylate, cyclopentyl diacrylate, hexyl diacrylate, cyclohexyl diacrylate, isobornyl diacrylate, vinyl diacrylate, allyl diacrylate, tetrahydrofurfuryl diacrylate, tris(2-hydroxy ethyl) isocyanurate diacrylate, benzyl acrylate, 2-phenylethyl acrylate, phenoxy acrylate, 2-phenoxyethyl acrylate, 2-hydroxy-3-phenoxypropyl acrylate, 2-(phenylthiol)ethyl acrylate, stearyl acrylate, alkoxy-EO (1-60) acrylates, hydroxy-EO (1-60) acrylates, alkoxy-PO (1-10) acrylates, alkoxy-PO (1-10) acrylates, phenol-EO (1-60) acrylates, phenol-PO (1-10) acrylates; the group consisting of methyl dimethacrylate, ethyl dimethacrylate, propyl dimethacrylate, butyl dimethacrylate, lauryl dimethacrylate, palmitoyl dimethacrylate, stearyl dimethacrylate, behenyl dimethacrylate, pentyl dimethacrylate, cyclopentyl dimethacrylate, hexyl dimethacrylate, cyclohexyl dimethacrylate, isobornyl dimethacrylate, vinyl dimethacrylate, allyl dimethacrylate, tetrahydrofurfuryl dimethacrylate, tris(2-hydroxy ethyl) isocyanurate dimethacrylate, benzyl methacrylate, 2-phenylethyl methacrylate, phenoxy methacrylate, 2-phenoxyethyl methacrylate, 2-hydroxy-3-phenoxypropyl methacrylate, 2-(phenylthiol)ethyl methacrylate, stearyl methacrylate, alkoxy-EO (1-60) methacrylates, hydroxy-EO (1-60) methacrylates, alkoxy-PO (1-10) methacrylates, hydroxy-EO (1-60) methacrylates, alkoxy-PO (1-10) methacrylates, phenol-EO (1-60) methacrylates, phenol-PO (1-10) methacrylates; the group consisting of glycerol diacrylate, EO (1-200)diacrylate, PO (1-200)diacrylate, 1,3-propanediol diacrylate, butanediol diacrylate, pentanediol diacrylate, neopentyl glycol diacrylate, hexanediol diacrylate, 2,2,4-trimethyl hexanediol diacrylate, 4,2,2-trimethyl hexanediol diacrylate, 1,4-cyclohexanediol diacrylate, dipropyleneglycol diacrylate, nonanediol diacrylate, decanediol diacrylate, dodecanediol diacrylate, 2,2-Bis [4-(2-hydroxy-3-acryloxypropoxy)phenyl]propane, tris(2-hydroxy ethyl) isocyanurate triacrylate, glycerol triacrylate, trimethylolpropane trimethylolpropaneEO(2-9)triacrylate, triacrylate, trimethylolethane triacrylate, 1,2,4-butanetriol triacrylate, pentaerythritol triacrylate, pentaerythritol tetraacrylate, pentaerythritol tetraacrylate, sorbitol hexaacrylate, dipentaerythritol pentaacrylate, dipentaerythritol hexaacrylate, ditrimethylolpropane tetraacrylate, 7,7,9-trimethyl-4, 13-dioxo-3,14 dioxa-5, 12-diazahexadecane-1, 16-diol-diacrylate; the group consisting of bisphenol-A-PO (2-10)dimethacrylate, bisphenol-A-EO (2-30)dimethacrylate, bisphenol-F-EO (2-10)dimethacrylate, bisphenol-F—PO (1-10)dimethacrylate, glycerol dimethacrylat, EO (1-200)dimethacrylate, PO (1-200)dimethacrylate, 1,3-propanediol dimethacrylate, butanediol dimethacrylate, pentanediol dimethacrylate, neopentyl glycol dimethacrylate, hexanediol dimethacrylate, 2,2,4- and 4,2,2-trimethyl hexanediol dimethacrylate, 1,4-cyclohexanediol dimethacrylate, dipropyleneglycol dimethacrylate, nonanediol dimethacrylate, decanediol dimethacrylate, dodecanediol dimethacrylate, 2,2-Bis [4-(2-hydroxy-3-methacryloxypropoxy)phenyl]propane, tris(2-hydroxy ethyl) isocyanurate trimethacrylate, glycerol trimethacrylate, trimethylolpropane trimethacrylate, trimethylolpropaneEO (2-9)trimethacrylate, trimethylolethane trimethacrylate, 1,2,4-butanetriol trimethacrylate, pentaerythritol trimethacrylate, pentaerythritol tetramethacrylate, pentaerythritol tetramethacrylate, sorbitol hexamethacrylate, dipentaerythritol pentamethacrylate, dipentaerythritol hexamethacrylate, 7,7,9-trimethyl-4, 13-dioxo-3, 14 dioxa-5, 12-diazahexadecane-1,16-diol-dimethacrylate, 7,9,9-trimethyl-4, 13-dioxo-3, 14 dioxa-5, 12-diazahexadecane-1,16-diol-dimethacrylate; the group consisting of bisphenol-A-PO (2-10)diacrylate, bisphenol-A-EO (2-30)diacrylate, bisphenol-F-EO (2-10)diacrylate, bisphenol-F—PO (1-10)diacrylate, glycerol dimethacrylat, EO (1-200)diacrylate, PO (1-200)diacrylate, 1,3-propanediol diacrylate, butanediol diacrylate, pentanediol diacrylate, neopentyl glycol diacrylate, hexanediol diacrylate, 2,2,4- and 4,2,2-trimethyl hexanediol diacrylate, 1,4-cyclohexanediol diacrylate, dipropyleneglycol diacrylate, nonanediol diacrylate, decanediol diacrylate, dodecanediol diacrylate, 2,2-Bis [4-(2-hydroxy-3-methacryloxypropoxy)phenyl]propane, tris(2-hydroxy ethyl) isocyanurate trimethacrylate, glycerol trimethacrylate, trimethylolpropane trimethacrylate, trimethylolpropaneEO (2-9)trimethacrylate, trimethylolethane trimethacrylate, 1,2,4-butanetriol trimethacrylate, pentaerythritol trimethacrylate, pentaerythritol tetramethacrylate, pentaerythritol tetramethacrylate, sorbitol hexamethacrylate, dipentaerythritol pentamethacrylate, 7,7,9-trimethyl-4, 13-dioxo-3,14 dioxa-5,12-dipentaerythritol hexamethacrylate, diazahexadecane-1, 16-diol-diacrylate, 7,9,9-trimethyl-4, 13-dioxo-3, 14 dioxa-5, 12-diazahexadecane-1, 16-diol-diacrylate.

In certain embodiments, the monomer, oligomer and/or polymer is selected from
bisphenol-A-EO (2-30)diacrylate, bisphenol-A-EO (2-30)dimethacrylate,
2-phenoxyethyl acrylate, 2-phenoxyethyl methacrylate,
butanediol diacrylate, butanediol dimethacrylate, hexandiol diacrylate, hexandiol dimethacrylate, nonanediol diacrylate, nonanediol dimethacrylate, decanediol diacrylate, decanediol dimethacrylate, dodecandiol diacrylate, dodecandiol dimethacrylate, cyclohexandiol dimethacrylate, cyclohexandiol diacrylate and
7,7,9-trimethyl-4, 13-dioxo-3, 14 dioxa-5, 12-diazahexadecane-1, 16-diol-dimethacrylate, 7,9,9-trimethyl-4, 13-dioxo-3, 14 dioxa-5, 12-diazahexadecane-1, 16-diol-dimethacrylate, 7,7,9-trimethyl-4, 13-dioxo-3, 14 dioxa-5, 12-diazahexadecane-1, 16-diol-dimethacrylate, 7,9,9-trimethyl-4, 13-dioxo-3, 14 dioxa-5, 12-diazahexadecane-1, 16-diol-dimethacrylate, 4,4′-methylendicyclohexyl dimethacrylate, 4,4′-methylendicyclohexyl diacrylate, tetramethylxylylene dimethacrylate, tetramethylxylylene diacrylate, toluenediol dimethacrylate, toluendiol diacrylate.

In a further certain embodiment of the first aspect of the invention, the oligomer is formed of up to 30 monomers.

The mixture according to any of the preceding claims, wherein the oligomer or polymer has a viscosity of at least 250 Pas at 60° C.

In certain embodiments, the mixture comprises a photoinitiator, wherein the photoinitiator concentration is 0.05 to 4 wt %, particularly the photoinitiator concentration is 1 to 3 wt %. In certain embodiments, the resin comprises a photoinitiator, wherein the photoinitiator concentration is 0.05 to 4 wt %. In certain embodiments, the resin comprises a photoinitiator, wherein the photoinitiator concentration is 0.05 to 3.5 wt %. In certain embodiments, the resin comprises a photoinitiator, wherein the photoinitiator concentration is 0.05 to 3 wt %. In certain embodiments, the resin comprises a photoinitiator, wherein the photoinitiator concentration is 1 to 4 wt %. In certain embodiments, the resin comprises a photoinitiator, wherein the photoinitiator concentration is 1 to 3.5 wt %. In certain embodiments, the resin comprises a photoinitiator, wherein the photoinitiator concentration is 1 to 3. wt %.

In certain embodiments, the photoinitiator is
a compound selected from the group of benzoines and benzoin ethers, in particular benzoin, benzoin methyl ether, benzoin ethyl ether and benzoin isopropyl ether, benzoin phenyl ether and benzoin acetate,
a compound selected from the group of acetophenones, in particular acetophenone, 2,2-dimethoxyacetophenone and 1,1-dichloroacetophenone,
a compound selected from the group of benzils and benzil ketals, in particular benzil, benzildimethyl ketal and benzildiethyl ketal,
a compound selected from the group of anthraquinones, in particular 2-methylanthraquinone, 2-ethylanthraquinone, 2-tert-butylanthraquinone, 1-chloroanthraquinone and 2-amylanthraquinone,
a compound selected from the group of triphenylphosphines and benzoylphosphine oxides, in particular triphenylphosphines, (2,4,6-trimethylbenzoyl)diphenylphosphine oxide (luzirin TPO) and bis(2,4,6-trimethylbenzoylphenyl)phosphine oxide,
a compound selected from the group of benzophenones, in particular benzophenone, 4, 4′-bis-(N, N′-dimethylamino)-benzophenone and 2-hydroxy-2-methylpropiophenone,
a compound selected from the group of thioxanthones and xanthones, in particular thioxanone and xanthone,
an acridine derivative, phenazine derivative, quinoxaline derivative
1-phenyl-1,2-propanedione-2-O-benzoyloxime,
a compound selected from the group of 1-aminophenyl ketones or 1-hydroxyphenyl ketones, in particular 1-hydroxycyclohexylphenyl ketone, phenyl (1-hydroxyisopropyl) ketone and 4-isopropylphenyl (1-hydroxyisopropyl) ketone.

Furthermore, the person skilled in the art can use a photoinitiator from US2008/0076847 A1, which is herein incorporated by reference. Additional photoinitiators are disclosed in WO2017178383 A1, EP1923406 A1 and WO2007018287 A1, which are incorporated by reference as well.

In certain embodiments, the photoinitiator is selected from the group consisting of bis(2,4,6-trimethylbenzoylphenyl)phosphine oxide, (2,4,6-trimethylbenzoyldiphenyl)phosphine oxide, 2-hydroxy-2-methylpropiophenone and 1-hydroxycyclohexylphenyl ketone or a resin thereof.

In certain embodiments, the resin comprises one or more additives selected from a UV absorber, an optical brightener and/or a colour pigment.

In certain embodiments, the optical brightener is selected from the group consisting of 2,5-bis-(5-tert-butyl-2-benzoxazolyl)thiophene, 4,4′-bis(2-methoxystyryl)-1,1′-biphenyl, 2,2,6,6-tetramethyl-4-piperidinol, bis(2,2,6,6, -tetramethyl-4-piperidyl) sebacate, bis(1,2,2,6,6-pentamethyl-4-piperidyl) sebacate and methyl-(1,2,2,6,6-pentamethyl-4-piperidyl) sebacate, decanedioic acid, bis(2,2,6,6-tetramethyl-1-(octyloxy)-4-piperidinyl) ester, bis(1,2,2,6,6-pentamethyl-4-piperidinyl)-[[3,5-bis(1, 1-dimethylethyl)-4-hydroxyphenyl]methyl]butylmalonate or mixtures thereof.

In certain embodiments, the UV stabilizer is selected from the group consisting of 2-isopropylthioxanthone, 1-phenylazo-2-naphtol, 2,5-bis-(5-tert-butyl-2-benzoxazolyl)thiophene, 4,4′-bis(2-methoxystyryl)-1,1′-biphenyl, 2,2,6,6-tetramethyl-4-piperidinol, bis(2,2,6,6, -tetramethyl-4-piperidyl) sebacate, bis(1,2,2,6,6-pentamethyl-4-piperidyl) sebacate and methyl-(1, 2, 2, 6, 6-pentamethyl-4-piperidyl) sebacate, decanedioic acid, bis(2,2,6,6-tetramethyl-1-(octyloxy)-4-piperidinyl) ester, bis(1,2,2,6,6-pentamethyl-4-piperidinyl)-[[3,5-bis(1, 1-dimethylethyl)-4-hydroxyphenyl]methyl]butylmalonate or mixtures thereof.

In another certain embodiment, the resin according to the first aspect of the invention is a resin for medical engineering.

A second aspect of the invention relates to a polymer, particularly produced by 3D printing using the resin for 3D printing according to claims 1 to 10, wherein the polymer comprises
polymerised monomers, oligomers or polymers, or a mixture thereof, comprising at least one acrylate or methacrylate subunit, and
a lactam,
   characterized in that the lactam concentration is 0.001 to 0.3 wt %, particularly the lactam concentration is 0.005 to 0.15 wt %, more particularly the lactam concentration is 0.01 to 0.1 wt %, and wherein the monomer, oligomer, polymer and lactam are defined as above. The lactam concentration can for example comprise any lactam concentration according to the first aspect of the invention.

In an certain embodiments, the polymer further comprises at least one photoinitiator, wherein the photoinitiator concentration is 0.05 to 4 wt %, particularly the photoinitiator concentration is 1 to 3 wt %. The photoinitiator can for example comprise any photoinitiator concentration according to the first aspect of the invention.

A third aspect of the invention relates to a dental polymer or an otoplastic polymer, wherein the dental polymer or otoplastic polymer is a polymer according to the first and/or second aspect of the invention.

Dental polymers are used for splints, milling blanks, as well as for restoring and replacing tooth structure and missing teeth. Otoplastic polymers are used for hearing-aid acoustics, including hearing aids or hearing protection.

A fourth aspect of the invention relates to a medical product according to the first, second and third aspect of the invention.

The invention is further illustrated by the following examples and figures, from which further embodiments and advantages can be drawn. These examples are meant to illustrate the invention but not to limit its scope.

### DESCRIPTION OF THE FIGURES

FIG. 1 penetration depth (Dp/μm) in dependency of the CL-Lactam-concentration (ppm).

FIG. 2 critical energy dosage (Ec/mJ/cm²) in dependency of the CL-Lactam-concentration (ppm).

FIG. 3 penetration depth (Dp/μm) in dependency of the Lactam-concentration (ppm).

FIG. 4 critical energy dosage (Ec/mJ/cm²) in dependency of the Lactam-concentration (ppm).

FIG. 5 penetration depth (Dp/μm) in dependency of the photoinitiator-concentration (ppm).

FIG. 6 critical energy dosage (Ec/mJ/cm²) in dependency of the photoinitator-concentration (ppm).

FIG. 7 penetration depth (Dp/μm) in dependency of the scavanger-concentration (ppm).

FIG. 8 critical energy dosage (Ec/mJ/cm²) in dependency of the scavanger-concentration (ppm).

FIG. 9 penetration depth (Dp/μm) in dependency of the brightener-concentration (ppm).

FIG. 10 Critical energy dosage (Ec/mJ/cm²) in dependency of the brightener-concentration (ppm).

FIG. 11 penetration depth (Dp/μm) in dependency of the UV-stabilizer-concentration (ppm).

FIG. 12 critical energy dosage (Ec/mJ/cm²) in dependency of the UV-stabilizer-concentration (ppm).

FIG. 13 3D test specimen with 1 mm and 2 mm bore in vertical and horizontal orientation.

FIG. 14 reduction of biofilm by up to 98% using a lactam modified resin.

FIG. 15 surface without added lactam, enlarged (A) and surface with added lactam, enlarged (B).

### EXAMPLES

### Example 1: Influence of Lactam on the Critical Energy and Depth of Cure of 3D Printing Material

A base formulation was prepared by dissolving the photo initiator in the oligomers by use of a laboratory stirring device (IKA RW20D) at 300 rotations per minute using a stainless-steel wing stirrer.

TABLE 1
Composition of the resin used as the base formulation.

| | Functional description of the | | | |
|---|---|---|---|---|
| No. | component | CAS | m-% | comments |
| 1 | Ethoxylated Bisphenol-A- | 41637-38-1 | 98 | mix of |
| | Dimethacrylates | | | ethoxylations |
| 2 | Photoinitiator | 84434-11-7 | 2 | |

Different proportions of the respective lactams were added to this base mixture and the critical energy and depth of cure were determined. The determination of critical energy (Ec) and depth of cure (Dp) were performed for each test compound as follows:

In a first step the light intensity of the test system (here: ASIGA Max UV 385) was measured using a calibrated radiometer. The light intensity is required to calculate the penetration depth and cure energy as it is described in the Paul Jacob's formula (Rapid Prototyping & Manufacturing: Fundamentals of StereoLithography). This can be done by simply project the light on the printing area and place the radiometer on every corner and on the middle of the printing area. In a second step the ASIGA composers point exposer was set to 5 mm radius. This point was used to point the light in a certain form to the liquid resin. In a third step the exposure time on composers point exposer was set and sent to the exposure plane equipped with a foil containing enough test compound. This test was carried out 4 times to obtain statistical relevance. In a fourth step the thickness of the projected circular object was determined with a calibrated caliper. In step 5, steps 2-4 were repeated for at least 5 different curing times to measure the cure depth within different lengths of exposure time. The interval of the exposure time varied depending on the materials used. Exposure times were successively shortened starting with longer exposures. In step 6, having measured the light intensity and cure depth within an interval of exposure time, Dp and Ec value was then calculated (Paul Jacob's, Rapid Prototyping & Manufacturing: Fundamentals of StereoLithography).

Lactam 1:4-(4-chlorophenyl)-5-methylene-pyrrol-2-one

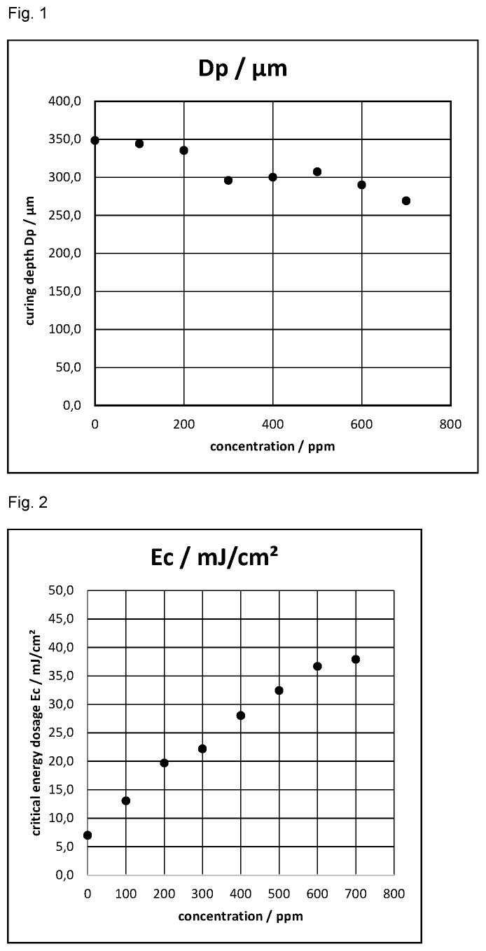

For curing depth and critical energy dosage see FIGS. 1 and 2.

TABLE 2
Penetration depth (Dp) and critical energy dosage (Ec) for
different concentrations of CI-Lactam in the base
formulation (Table 1).

| No. | CI-Lactam, ppm | Dp, μm | Ec, mJ/cm\hat{ }2 |
|---|---|---|---|
| 1 | 0 | 348.6 | 7 |
| 2 | 100 | 344.2 | 13.1 |
| 3 | 200 | 335.4 | 19.7 |
| 4 | 300 | 296.2 | 22.2 |
| 5 | 400 | 300.4 | 28.1 |
| 6 | 500 | 307.4 | 32.4 |
| 7 | 600 | 288.3 | 36.7 |
| 8 | 700 | 269.1 | 37.9 |

Lactam 2:5-methylene-4-(p-tolyl)-1H-pyrrol-2 (5H)-one (CAS 2084047-27-6)

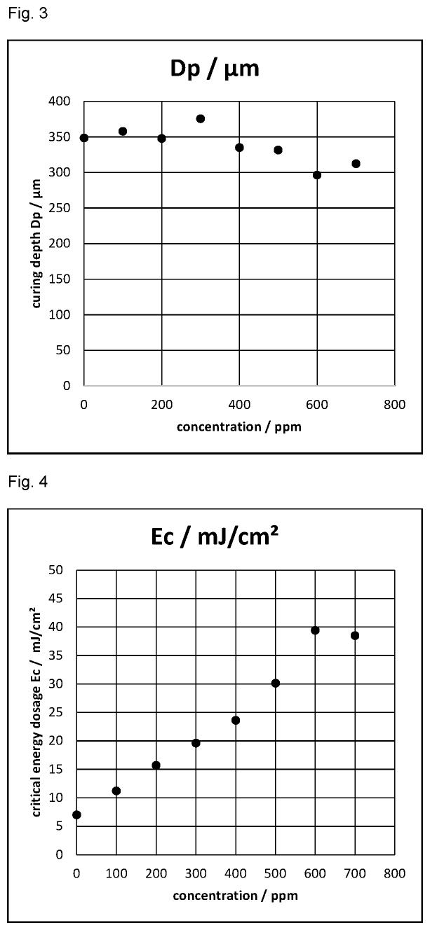

For curing depth and critical energy dosage see FIGS. 3 and 4.

TABLE 3
Penetration depth (Dp) and critical energy dosage (Ec) tor
different concentrations of Lactam in the base formulation
(Table 1).

| No. | Lactam, ppm | Dp, μm | Ec, mJ/cm\hat{ }2 |
|---|---|---|---|
| 1 | 0 | 348.6 | 7 |
| 2 | 100 | 357.9 | 11.2 |
| 3 | 200 | 348.7 | 15.7 |
| 4 | 300 | 375.6 | 19.6 |
| 5 | 400 | 335.1 | 23.6 |
| 6 | 500 | 331.6 | 30.1 |
| 7 | 600 | 269.3 | 39.4 |
| 8 | 700 | 312.3 | 38.5 |

### Example 2: Influence of Photo-Initiator Concentration on the Critical Energy Dosage and Depth of Cure of 3D Printing Material

A base formulation was prepared by dissolving the lactam in the oligomers by use of a laboratory stirring device (IKA RW20D) at 300 rotations per minute using a stainless-steel wing stirrer.

TABLE 4
Composition of the resin used as the base formulation.

| | Functional description of the | | | |
|---|---|---|---|---|
| No. | component | CAS | m-% | comments |
| 1 | Ethoxylated Bisphenol-A- | 41637-38-1 | 99.95 | mix of |
| | Dimethacrylates | | | ethoxylations |
| 2 | Lactam | 2084047-27- | 0.05 | |
| | | 6 | | |

Ethyl Penyl(2,4,6-trimethylbenzoyl)phosphinate (CAS 84434-11-7)

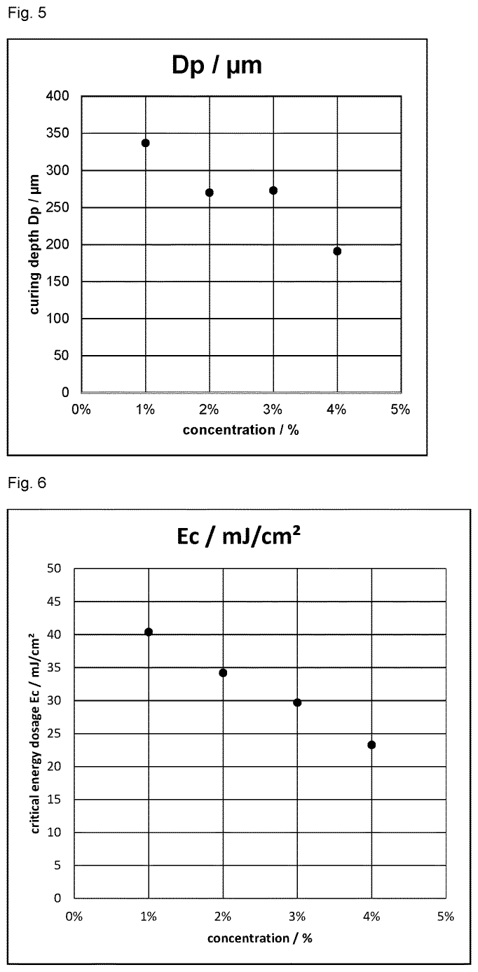

For curing depth and critical energy dosage see FIGS. 5 and 6.

TABLE 5
Penetration depth (Dp) and critical energy dosage (Ec) for different
concentrations of photoinitiator (PI) in the base formulation (Table 4).

| No. | PI, m-% | Dp, μm | Ec, mJ/cm\hat{ }2 |
|---|---|---|---|
| 1 | 1 | 337 | 40.4 |
| 2 | 2 | 270 | 34.2 |
| 3 | 3 | 273 | 29.7 |
| 4 | 4 | 191 | 23.3 |

### Example 3: Influence of Anaerobic Radical Scavenger on the Critical Energy and Depth of Cure of 3D Printing Material

A base formulation was prepared by dissolving the photo initiator in the oligomers by use of a laboratory stirring device (IKA RW20D) at 300 rotations per minute using a stainless-steel wing stirrer.

TABLE 6
Composition of the resin used as the base formulation.

| | Functional description of the | | | |
|---|---|---|---|---|
| No. | component | CAS | m-% | comments |
| 1 | Ethoxylated Bisphenol-A- | 41637-38-1 | 98 | mix of |
| | Dimethacrylates | | | ethoxylations |
| 2 | Photoinitiator | 84434-11-7 | 2 | |

Different proportions of anaerobic radical scavenger (CAS 2564-83-2) were added to this base mixture and the critical energy and depth of cure were determined. The determination of critical energy (Ec) and depth of penetration (Dp) were performed for each test compound described in Example 1.

2,2,6,6-Tetramethylpiperidine 1-Oxyl Free Radical (CAS 2564-83-2)

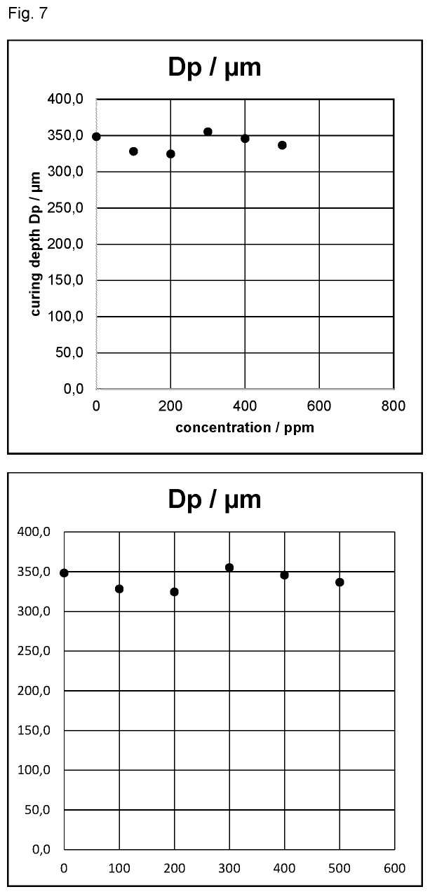

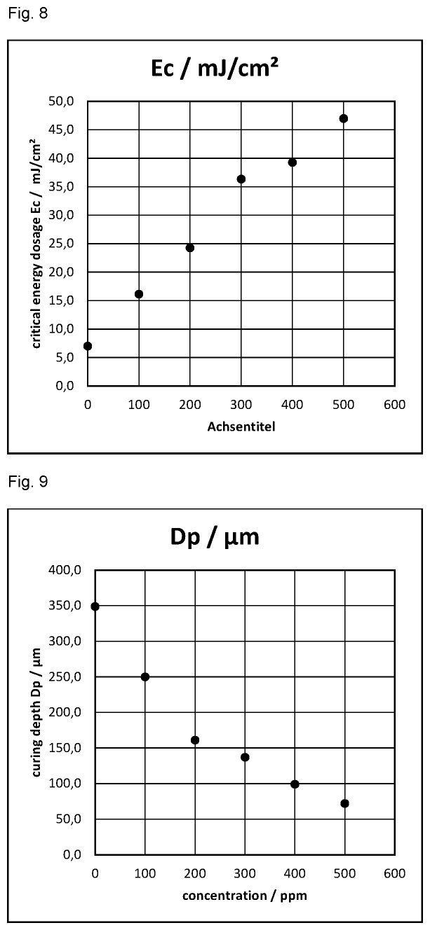

For curing depth and critical energy dosage see FIGS. 7 and 8.

TABLE 7
Penetration depth (Dp) and critical energy dosage (Ec) for different
concentrations of scavanger in the base formulation (Table 6).

| No. | Scavenger, ppm | Dp, μm | Ec, mJ/cm\hat{ }2 |
|---|---|---|---|
| 1 | 0 | 348.6 | 7 |
| 2 | 100 | 328.4 | 16.1 |
| 3 | 200 | 324.7 | 24.3 |
| 4 | 300 | 355.3 | 36.4 |
| 5 | 400 | 345.9 | 39.3 |
| 6 | 500 | 336.8 | 47 |

### Example 4: Influence of Optical Brightener on the Critical Energy and Depth of Cure of 3D Printing Material

A base formulation was prepared by dissolving the photo initiator in the oligomers by use of a laboratory stirring device (IKA RW20D) at 300 rotations per minute using a stainless-steel wing stirrer.

TABLE 8
Composition of the resin used as the base formulation.

| | Functional description of the | | | |
|---|---|---|---|---|
| No. | component | CAS | m-% | comments |
| 1 | Ethoxylated Bisphenol-A- | 41637-38-1 | 98 | mix of |
| | Dimethacrylates | | | ethoxylations |
| 2 | Photoinitiator | 84434-11-7 | 2 | |

Different proportions of optical brightener (CAS 7128-64-5) were added to this base mixture and the critical energy and depth of cure were determined. The determination of critical energy (Ec) and depth of penetration (Dp) were performed for each test compound described in example 1.

2,5-Bis(5-tert-butyl-2-benzoxazolyl)thiophene (CAS 7128-64-5)

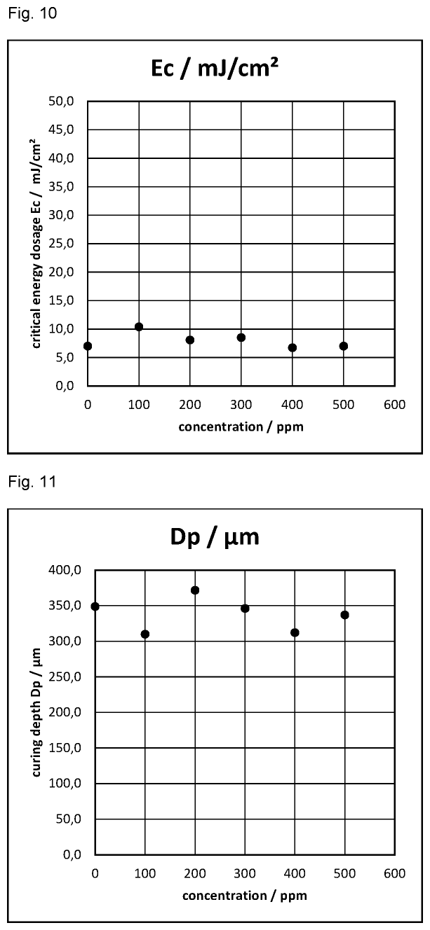

For curing depth and critical energy dosage see FIGS. 9 and 10.

TABLE 9
Penetration depth (Dp) and critical energy dosage (Ec) for different
concentrations of brightener in the base formulation (Table 8).

| No. | Brightener, ppm | Dp, μm | Ec, mJ/cm\hat{ }2 |
|---|---|---|---|
| 1 | 0 | 348.6 | 7 |
| 2 | 100 | 328.4 | 16.1 |
| 3 | 200 | 324.7 | 24.3 |
| 4 | 300 | 355.3 | 36.4 |
| 5 | 400 | 345.9 | 39.3 |
| 6 | 500 | 336.8 | 47 |

### Example 5: Influence of UV Stabilizer on the Critical Energy and Depth of Cure of 3D Printing Material

A base formulation was prepared by dissolving the photo initiator in the oligomers by use of a laboratory stirring device (IKA RW20D) at 300 rotations per minute using a stainless-steel wing stirrer.

TABLE 10
Composition of the resin used as the base formulation.

| | Functional description of the | | | |
|---|---|---|---|---|
| No. | component | CAS | m-% | comments |
| 1 | Ethoxylated Bisphenol-A- | 41637-38-1 | 98 | mix of |
| | Dimethacrylates | | | ethoxylations |
| 2 | Photoinitiator | 84434-11-7 | 2 | |

Different proportions of UV stabilizer (CAS 2440-22-4) were added to this base mixture and the critical energy and depth of cure were determined. The determination of critical energy (Ec) and depth of penetration (Dp) were performed for each test compound described in example 1.

2-(2-Hydroxy-5-methylphenyl)benzotriazole (CAS 2440-22-4)

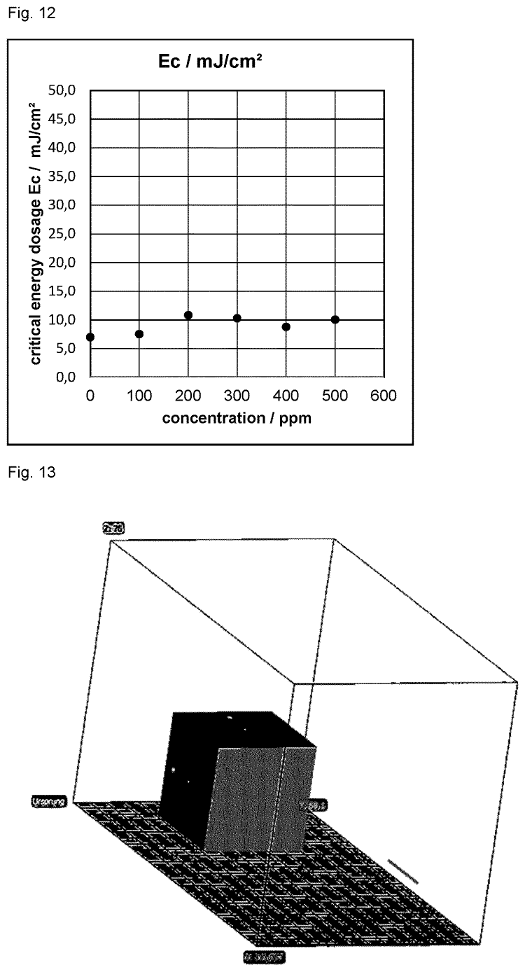

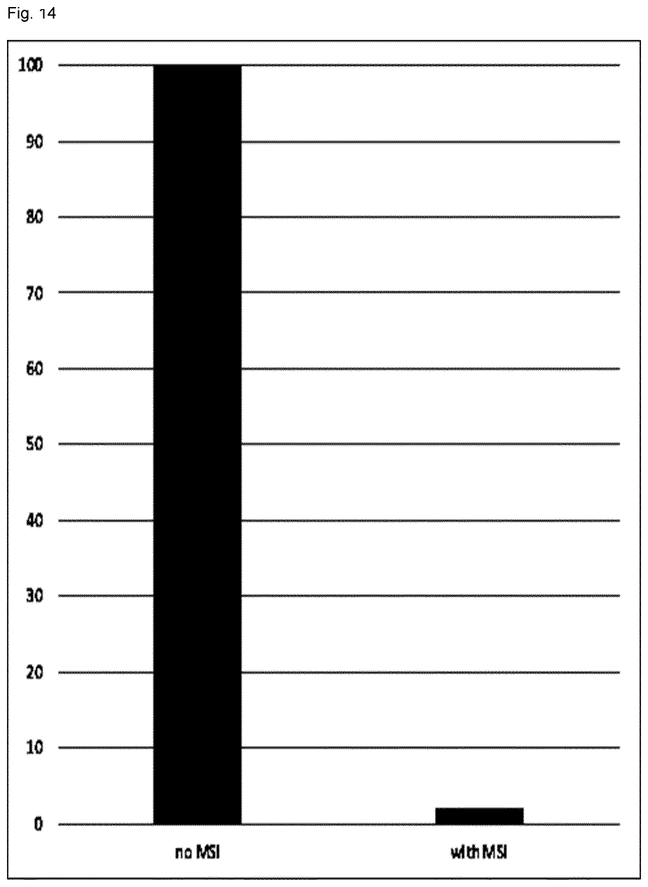

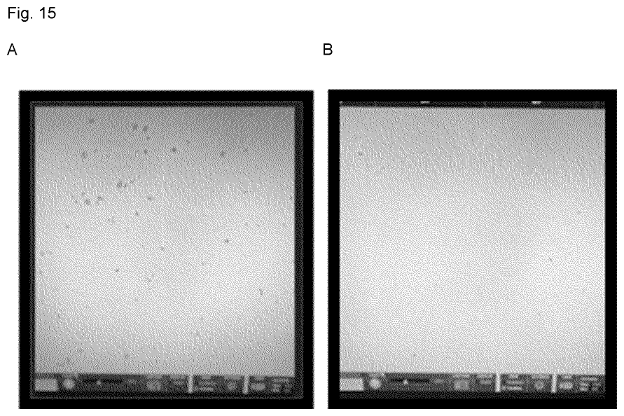

For curing depth and critical energy dosage see FIGS. 11 and 12.

TABLE 11
Penetration depth (Dp) and critical energy dosage (Ec) for different
concentrations of UV-stabilizer in the base formulation (Table 10).

| | UV stabilizer, | | |
|---|---|---|---|
| No. | ppm | Dp, μm | Ec, mJ/cm\hat{ }2 |
| 1 | 0 | 348.6 | 7 |
| 2 | 100 | 309.7 | 7.5 |
| 3 | 200 | 371.5 | 10.8 |
| 4 | 300 | 345.9 | 10.3 |
| 5 | 400 | 312.0 | 8.8 |
| 6 | 500 | 336.8 | 10.0 |

### Example 6: 3D Printing Accuracy of a Lactam Modified Resin Compared to Standard Material Standard Resin

TABLE 12
Formulation of standard resin.

| | Functional description | | | |
|---|---|---|---|---|
| No. | of the component | CAS | m-% | comments |
| 1 | Ethoxylated Bisphenol-A- | 41637-38- | 97.94 | mix of |
| | Dimethacrylates | 1 | | ethoxylations |
| 2 | Photoinitiators | 84434-11- | 2 | |
| | | 7 | | |
| 3 | UV absorber | 2440-22-4 | 0.05 | |
| 4 | Tinopal OB | 7128-64- | 0.01 | |
| | | 5 | | |

Dp = 205.7 μm
Ec = 10.5 mJ/cm²

### Lactame Modified Resin

TABLE 13
Formulation of lactam modified resin.

| | Functional description of the | | | |
|---|---|---|---|---|
| No. | component | CAS | m-% | comments |
| 1 | Ethoxylated Bisphenol-A- | 41637-38-1 | 97.89 | mix of |
| | Dimethacrylates | | | ethoxylations |
| 2 | Photoinitiators | 84434-11-7 | 2 | |
| 3 | UV absorber | 2440-22-4 | 0.05 | |
| 4 | Optical Brightener | 7128-64-5 | 0.01 | |
| 5 | Lactam | 2084047-27- | 0.05 | |
| | | 6 | | |

Dp = 99.6 μm
Ec = 11.6 mJ/cm²

Test specimen for determining the 3D printing accuracy

The test specimen comprises a top surface with a 1 mm hole and 2 mm hole in 0° inclination (alignment to radiation direction). Furthermore, the left surface of the test specimen comprises a 1 mm hole and 2 mm hole in 90° inclination (see FIG. 13). With increasing inclination to the radiation direction, effects that adversely affect 3D printing accuracy (e.g. z overexposure, xy scattering) increasingly occur. The quality of the 3D printing result allows conclusions to be drawn about how well a 3D printing material can be adjusted against the above-mentioned effects. Both a lactam-modified material and a standard material were generated with 3D printing (Slicer used: ASIGA Composer). The quality of the generated bore geometries was microscopically examined and evaluated.

Thus, the lactam-modified material shows significantly better drawing accuracy compared to the standard material. As expected, the bore geometries in alignment of the optical path are comparably good (z overexposure cannot occur here).

### Example 7: Influence of Lactam on Mechanical Properties

To exclude possible adverse effects of the lactam on the mechanical properties of the material, the formulations described in Example 6 were subjected to a mechanics test following ISO 178.

TABLE 14
Mechanical data of standard resin, tested against ISO 178.

| | Flexural | Flexural | Elongation at |
|---|---|---|---|
| | Modulus, MPa | Strength, MPa | Break, % |
| Average | 2343 | 113 | 10 |
| Std. Deviation | 84 | 6 | 2.8 |
| Number of samples | 10 | 10 | 10 |

TABLE 15
Mechanical data of lactam modified resin, tested against ISO 178.

| | Flexural | Flexural | Elongation at |
|---|---|---|---|
| | Modulus, MPa | Strength, MPa | Break, % |
| Average | 2260 | 104 | 9.7 |
| Std. Deviation | 97 | 6.5 | 3.2 |
| Number of samples | 10 | 10 | 10 |

From table 14 and 15 one can observe that the lactam-modified material shows equal mechanical performance compared to the standard material.

### Example 8: Conclusions

Varying PI concentration decreases both Dp and Ec values, wherein low EC values have negative impact on shelf-life stability of resin. Radical scavenger increases Ec values and shows no influence on Dp values, wherein radical scavenger is not useful to regulate through-cure of resin. Optical brightener decreases Dp but shows no influence on Ec value, which is not useful to function as stabilizing agent. UV stabilizer shows no effect in reasonable concentrations and is not suitable to regulate curing characteristics of resin except using higher concentration, which should be avoided due to SVHC concerns of this type of substances. Lactam increases Ec value and furthermore decreases Dp value in parallel, which are advantageous properties to regulate curing characteristics of resin-optimal for high resolution printing materials as well as long-term stability in use.

1. A mixture, for 3D printing, particularly a resin for 3D printing comprising
a monomer, oligomer or polymer, or a mixture thereof, comprising at least one acrylate or methacrylate subunit, and
a lactam,
characterized in that the lactam concentration is 0.001 to 0.3 wt %, particularly the lactam concentration is 0.005 to 0.15 wt %, more particularly the lactam concentration is 0.01 to 0.1 wt %.

2. The mixture according to claim 1, wherein the lactam is a γ-lactam or furanone, particularly a γ-lactam.

3. The mixture according to claim 1, wherein the lactam is a compound of formula (1) or (2) 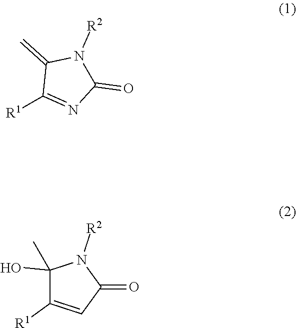
wherein R¹ is a substituted or unsubstituted aryl, wherein the substituted aryl is substituted with Z¹, wherein Z¹ is selected from the group consisting of C₁-C₃ alkyl F, Cl, and Br, and
R² is selected from H and the group consisting of —C₁₋₃-alkyl-N⁺Me₃, particularly R² is H.

4. The mixture according to claim 1, wherein the monomer is of formula (3a), the oligomer is of formula (3b) and the polymer is of formula (3c), 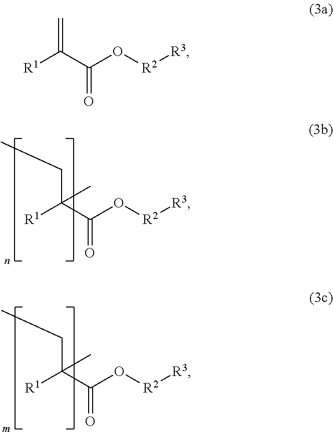
wherein
n is 2 to 30 and m is 31 to 100,
R¹ is —H or —CH₃,
R² is selected from the group consisting of
—C₁-bis C₂₂-alkyl-, -allyl-, -vinyl-,
—C₁-bis C₂₂-alkyl-O—C₁-bis C₂₂-alkyl-, wherein the —C₁₋₂₂-alkyl- is further substituted, particularly wherein the —C₁₋₂₂-alkyl- is further substituted with 1 to 4 acrylate or 1 to 4 methacrylate moieties,
—[(CH₂)ₑ—O-]_{f}-C₁₋₆-alkyl-[(CH₂)ₑ—O-]_{f}, wherein the —C₁₋₆-alkyl- is further substituted, particularly wherein the —C₁₋₆-alkyl- is further substituted with 1 to 4-[(CH₂)ₑ—O-]_{f}-acrylate moieties or 1 to 4-[(CH₂)ₑ—O-]_{f} methacrylate moieties, and wherein e is 1 to 3 and f is 2 to 9, 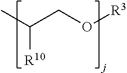
wherein j is 1 to 200, and R¹⁰ is selected from the group consisting of —H, —C₁₋₃-alkyl, —O—C₁₋₃-alkyl, —OH or -phenol,
—(C₁₋₃-alkyl)-O(C═O)NH—R¹¹—NH—C(═O)O—, wherein R¹¹ is a linker system consisting of the group aliphatic or aromatic moieties or a mixture thereof, 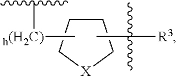
wherein X is O or CH and h is 0 or 1, 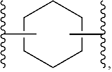
—Ph-Y—(C₁₋₃-alkyl)ₖ- wherein Y is S, O or CH₂ and k is 0 or 1 and wherein the C₁₋₃-alkyl may be further substituted with a moiety selected from the group consisting of —OH and NH₂, 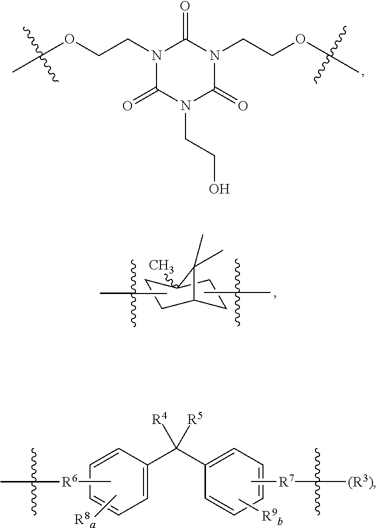
wherein
R⁴ and R⁵ are independently from each other selected from the group consisting of H, —C₁₋₄-alkyl, —CF₃ and phenyl, or
R⁴ and R⁵ form a ring structure consisting of 4 to 8 carbon atoms, particularly R⁴ and R⁵ are independently from each other selected from the group consisting of H and —C₁₋₄-alkyl
R⁶ and R⁷ are selected from —[(CH₂)ₚ—O-]_{q}-, and —O—[(CH₂)ᵥ—O-]_{w}-, wherein p and v are between 1 and 4, particularly m, p and v are between 1 and 3, q is between 1 and 4 and w is between 1 and 200, particularly w is between 1 and 60, more particularly w is between 1 and 30
Rₐ⁸ and R_{b}⁹ are independently from each other selected from the group consisting of-C₁₋₄-alkyl and phenyl,
a is 0 or 1, particularly a is 0
b is 0 or 1, particularly b is 0 and
R³ is selected from the group consisting of —H, acrylate and methacrylate.

5. The mixture according to claim 1, wherein R² is selected from
—C₁— bis C₂₂-alkyl-, -allyl-, -vinyl-,
—(C₁₋₃-alkyl)-O(C—O)NH—R¹¹—NH—C(═O)O—, wherein R¹¹ is a linker system consisting of the group aliphatic or aromatic moieties or a mixture thereof,
—Ph—Y—(C₁₋₃-alkyl)ₖ- wherein Y is S, O or CH₂ and k is 0 or 1 and wherein the C₁₋₃-alkyl may be further substituted with a moiety selected from the group consisting of —OH and NH₂, 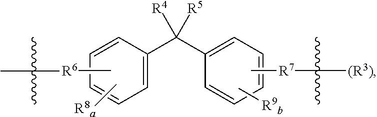
wherein
R⁴ and R⁵ are independently from each other selected from the group consisting of H, —C₁₋₄-alkyl, —CF₃ and phenyl, or
R⁴ and R⁵ form a ring structure consisting of 4 to 8 carbon atoms, particularly R⁴ and R⁵ are independently from each other selected from the group consisting of H and —C₁₋₄-alkyl
R⁶ and R⁷ are selected from —[(CH₂)ₚ—O-]_{q}-, and —O—[(CH₂)ᵥ—O-]_{w}-, wherein p and v are between 1 and 4, particularly m, p and v are between 1 and 3, q is between 1 and 4 and w is between 1 and 200, particularly w is between 1 and 60, more particularly w is between 1 and 30
R⁸ and R⁹% are independently from each other selected from the group consisting of-C₁₋₄-alkyl and phenyl,
a is 0 or 1, particularly a is 0
b is 0 or 1, particularly b is 0.

6. The mixture according to claim 1, wherein R² is selected from
—C₁-bis C₂₂-alkyl-, particularly C₁₋₁₃-alkyl,
—(C₁₋₃-alkyl)-O(C═O)NH—R¹¹—NH—C(═O)O—, wherein R¹¹ wherein R¹¹ is a linker system consisting of the group aliphatic or aromatic moieties or a mixture thereof, and 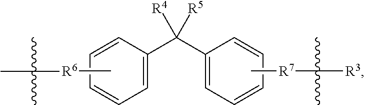
wherein
R⁴ and R⁵ are independently from each other selected from the group consisting of H, —C₁₋₄-alkyl,
R⁶ and R⁷ are selected from —[(CH₂)ₚ—O-]_{q}-, and —O—[(CH₂)ᵥ—O-]_{w}-, wherein p and v are between 1 and 4, particularly m, p and v are between 1 and 3, q is between 1 and 4 and w is between 1 and 200, particularly w is between 1 and 60, more particularly w is between 1 and 30.

7. The mixture according to claim 1, wherein R⁶ and R⁷ are selected from —O—[(CH₂)ᵥ—O-]_{w}-, wherein v is between 1 and 4, particularly v is between 1 and 3, and w is between 1 and 200, particularly w is between 1 and 60, more particularly w is between 1 and 30.

8. The mixture according to claim 1, wherein R¹¹ is selected from the group consisting of C₁₋₁₅ alkyl, and substituted or unsubstituted C₆ aryl, particularly R¹¹ is selected from the group consisting of C₁₋₁₃ alkyl, and substituted or unsubstituted C₆ aryl.

9. The mixture according to claim 1, wherein the oligomer is formed of up to 30 monomers.

10. The mixture according to claim 1, wherein the resin comprises a photoinitiator, wherein the photoinitiator concentration is 0.05 to 4 wt %, particularly the photoinitiator concentration is 1 to 3 wt %.

11. The resin according to claim 1, wherein the resin is a resin for medical engineering.

12. A polymer, particularly produced by 3D printing using the resin for 3D printing according to claim 1, wherein the polymer comprises
polymerised monomers, oligomers or polymers, or a mixture thereof, comprising at least one acrylate or methacrylate subunit, and
a lactam,
characterized in that the lactam concentration is 0.001 to 0.3 wt %, particularly the lactam concentration is 0.005 to 0.15 wt %, more particularly the lactam concentration is 0.01 to 0.1 wt %.

13. The polymer according to claim 12, wherein the polymer further comprises at least one photoinitiator, wherein the photoinitiator concentration is 0.05 to 4 wt %, particularly the photoinitiator concentration is 1 to 3 wt %.

14. A dental polymer or an otoplastic polymer, wherein the dental polymer or otoplastic polymer is a polymer according to claim 12.

15. A medical product, wherein the medical product comprises the polymer according to claim 12.
